(19) United States
(12) Patent Application Publication
(10) Pub. No.: US 2016/0031878 A1
(43) Pub. Date: Feb. 4, 2016

(54) RADIOLABELED COMPOUNDS

(71) Applicant: Hoffmann-La Roche Inc., Little Falls, NJ (US)

(72) Inventors: Edilio BORRONI, Basel (CH); Alexander FLOHR, Loerrach (DE); Luca GOBBI, Buus (CH); Thomas HARTUNG, Loerrach (DE); Raphael HOAREAU, Singapore (SG); Michael HONER, Zuerich (CH); Laurent MARTARELLO, Singapore (SG)

(73) Assignee: Hoffmann-La Roche Inc., Little Falls, NJ (US)

(21) Appl. No.: 14/813,293

(22) Filed: Jul. 30, 2015

Related U.S. Application Data
(63) Continuation of application No. PCT/EP2014/051180, filed on Jan. 22, 2014.

(30) Foreign Application Priority Data
Jan. 31, 2013 (EP) 13153404.2

(51) Int. Cl. C07D 471/04; A61K 51/04; C07B 59/00

(52) U.S. Cl. CPC C07D 471/04; C07B 59/002; A61K 51/0455; A61K 51/0463; C07B 2200/05

(57) ABSTRACT

The present invention relates to radiolabeled compounds of formula I

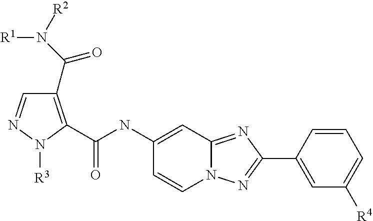

wherein R¹, R², R³ and R⁴ are as defined herein.

The present invention relates to radiolabeled compounds of formula I

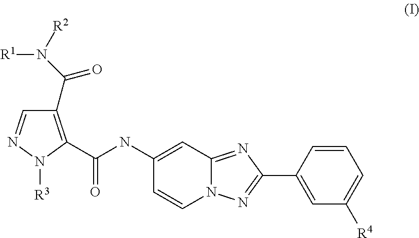

wherein

R¹ and R² are independently selected from C₁₋₇ alkyl, C₁₋₇haloalkyl, R¹ and R², together with the nitrogen atom to which they are attached, form heterocycloalkyl,

R³ is C₁₋₇ alkyl, R⁴ is hydrogen, C₁₋₇ alkoxy or C₁₋₇haloalkoxy and

wherein either R¹, R², R³ or R⁴ is labeled with a radionuclide selected from ³H, ¹¹C and ¹⁸F. The compounds of the present invention are useful for the labelling and diagnostic imaging of PDE10A functionality.

It has been found that the radiolabelled compounds of formula I may be used as PET (Positron Emission Tomography) and/or autoradiography radiotracer for the labelling and diagnostic molecular imaging of PDE10A functionality. Molecular imaging is based on the selective and specific interaction of a molecular probe (e.g. a radiotracer) with a biological target (for instance a receptor, an enzyme, an ion channel or any other cellular or extracellular component that is able to bind or retain the molecular probe) which is visualized through PET, nuclear magnetic resonance, near infrared or other methods. PET, a nuclear medical imaging modality, is ideally suited to produce three-dimensional images that provide important information on the distribution of a biological target in a given organ, or on the metabolic activity of such organ or cell or on the ability of a drug to enter such organ, bind to a biological target and/or modify biological processes. Since PET is a non-invasive imaging technique it can be used to investigate the pathophysiology of a disease and the action of drug on a given molecular target or cellular processes in humans and in animals. The availability of a PET radiotracer specific for a given molecular target can facilitate drug development and the understanding of the mechanism of action of a drug. In addition, a PET radiotracer may facilitate diagnosis of a disease by demonstrating pathophysiological changes taking place as a consequence of the disease. PDE10A is a dual substrate PDE encoded by a single gene as reported in 1999 by three separate research groups (Fujishige K., et al., Eur J Biochem (1999) 266(3):1118-1127, Soderling S. H., et al., ProcNatl Acad Sci USA (1999) 96(12):7071-7076, Loughney K., et al., Gene (1999) 234(1):109-117). PDE10A is unique from other members of the multigene family with respect to amino acid sequence (779 aa), tissue-specific pattern of expression, affinity for cAMP and cGMP and the effect on PDE activity by specific and general inhibitors.

PDE10A has one of the most restricted distributions of any PDE family being primarily expressed in the brain particularly in the nucleus accumbens and the caudate putamen. Additionally thalamus, olfactory bulb, hippocampus and frontal cortex show moderate levels of PDE10A expression. All these brain areas have been suggested to be involved in the pathophysiology of schizophrenia and psychosis, suggesting a central role of PDE10A in this devastating mental illness. Outside the central nervous system PDE10A transcript expression is also observed in peripheral tissues like thyroid gland, pituitary gland, insulin secreting pancreatic cells and testes (Fujishige, K. et al., J. Biol. Chem. 1999, 274, 18438-18445. Sweet, L. (2005) WO 2005/012485). On the other hand expression of PDE10A protein has been observed only in enteric ganglia, in testis and epididymal sperm (Coskran T. M., et al., J. Histochem. Cytochem. 2006, 54 (11), 1205-1213).

The human brain is a complex organ, consisting of millions of intercommunicating neurons. The understanding of abnormalities relating to diseases is the key to the future development of effective diagnosis and novel therapeutics. The study of biochemical abnormalities in human is rapidly becoming an essential and integral component of drug discovery and development process. Traditionally, the discovery and development of new drugs has been performed with a heavy emphasis on in vitro techniques to select promising lead candidates which are subsequently tested in living animals prior to human administration. Because in vitro systems reflect only part of the complexity of living systems and in vivo animal models of human disease are often only an approximation of human pathology, there is growing realization that a robust understanding of drug-receptor interaction in living man at an early stage in this process will be a major driving force in further enhancing the efficient and timely discovery and development of novel therapeutics. Over recent years, there has been a growing use of human medical imaging to assess pathologies, disease processes and drug action. These imaging modalities include PET, MRI, CT, ultrasound, EEG, SPECT and others (British Medical Bulletin, 2003, 65, 169-177). Therefore, the use of non-invasive imaging modalities, e.g. PET is an invaluable tool for the development of drugs in the future. Non-invasive nuclear imaging techniques can be used to obtain basic and diagnostic information about the physiology and biochemistry of a variety of living subjects. These techniques rely on the use of sophisticated imaging instrumentation that is capable of detecting radiation emitted from radiotracers administered to such living subjects. The information obtained can be reconstructed to provide planar and tomographic images that reveal distribution of the radiotracer as a function of time. The use of radiotracers can result in images which contain information on the structure, function and most importantly, the physiology and biochemistry of the subject. Much of this information cannot be obtained by other means. The radiotracers used in these studies are designed to have defined behaviors in vivo which permit the determination of specific information concerning the physiology or biochemistry of the subject. Currently, radiotracers are available for obtaining useful information concerning cardiac function, myocardial blood flow, lung perfusion, liver function, brain blood flow, regional brain glucose and oxygen metabolism, function of several brain receptors and enzymes and visualization of amyloid beta plaque deposits in Alzheimer's disease (PET Molecular Imaging and Its Biological Applications, Eds. Michael E. Phelps, Springer, New York, 2004. Ametamy S. et al., Chem. Rev., 2008, 108, 1501-1516. Nordberg A. et al. Nat. Rev. Neurol., 2010, 6, 78-87).

### Furthermore,

PET imaging provides a non-invasive and quantitative assay of normal and abnormal neurochemistry in human at an early stage of the drug development to enhance the efficient and effective discovery of therapeutics.

Tracer doses of labeled compounds enable the early evaluation of novel drugs: bio-distribution studies; receptor occupancy studies to optimize drug-dosing regime and characterizing downstream responses of drug action.

Understanding disease mechanisms in human using non-invasive techniques is intimately connected with future developments in the diagnosis and management of diseases and of novel therapeutics.

The radionuclides commonly used in PET include ¹¹C, ¹³N, ¹⁵O or ¹⁸F. In principle, it is possible to label all drugs by replacing one of the parent compound atoms with a PET nuclide, but only a few are found applicable as imaging agents in vivo in humans. The radioactive halftime of ¹¹C, ¹³N, ¹⁵O and ¹⁸F are 20, 10, 2 and 110 min, respectively. These short half-lives endow a number of advantages to their use as tracers to probe biological processes in vivo using PET. Repeat studies in the same subject within the same day are made possible. PET is being increasingly used as a tool to determine drug-dose-enzyme/receptor occupancy relationships in well-defined compounds. The use of PET radiotracers that specifically bind to the target receptor or enzyme can provide information about

the ability of a drug to enter the brain and bind to the target site,

the degree of occupancy of the target site produced by a given dose of drug,

the time-course of occupancy, and

the relative plasma and tissue kinetics of the drug in question.

Occupancy studies are performed with PET radiotracers which are usually not identical to the drug candidate under study (British Medical Bulletin, 2003, 65, 169-177).

Tritium labeled compounds are particularly valuable and widely used for studies involving high resolution autoradiography. The physical (nuclear) properties of tritium, the low maximum beta energy (18 keV) of the radiation and the high maximum specific activity (29 Ci/mg atom of hydrogen), makes tritium the ideal isotope for determining the precise localization of compounds, drugs and hormones for example, in biological specimens.

The present invention relates to radiolabeled compounds of formula I

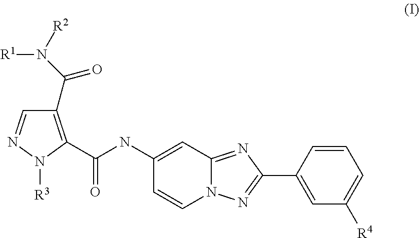

wherein

R¹ and R² are independently selected from C₁₋₇ alkyl, C₁₋₇ haloalkyl, R¹ and R², together with the nitrogen atom to which they are attached, form heterocycloalkyl,

R³ is C₁₋₇ alkyl,

R⁴ is hydrogen, C₁₋₇ alkoxy or C₁₋₇ haloalkoxy and

wherein either R¹, R³ or R⁴ is labeled with a radionuclide selected from ³H,

In a particular embodiment the invention relates to radiolabeled compounds of formula I, wherein

R¹ is C₁₋₇ alkyl,

R² is C₁₋₇ fluoroalkyl,

R³ is methyl,

R⁴ is hydrogen, wherein either R² is labeled with ¹⁸F or ³H, or R³ is labeled with ¹¹C.

In a particular embodiment the invention relates to radiolabeled compounds of formula I, wherein

R¹ and R² together with the nitrogen atom to which they are attached, form a heterocycloalkyl, preferably morpholinyl,

R³ is methyl,

R⁴ is C₁₋₇ fluoroalkyoxy, wherein R⁴ is labeled with ¹⁸F.

In a particular embodiment the invention relates to radiolabeled compounds of formula I, wherein

R¹ and R² together with the nitrogen atom to which they are attached, form a heterocycloalkyl, preferably morpholinyl,

R³ is methyl,

R⁴ is C₁₋₇ alkyoxy, wherein R⁴ is either labeled with ³H or ¹¹C.

In a particular embodiment the invention relates to radiolabeled compounds of formula I selected from the group consisting of:

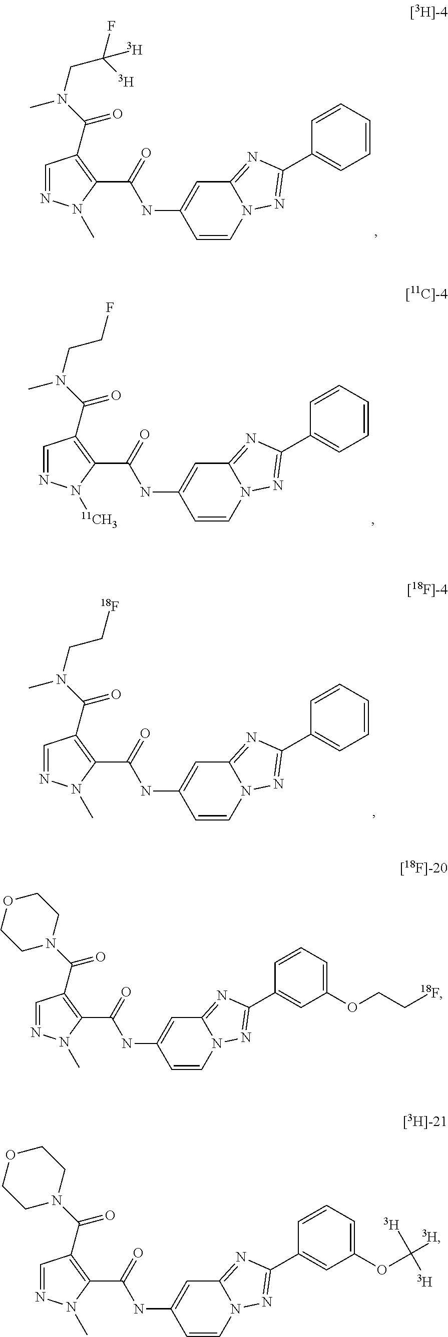

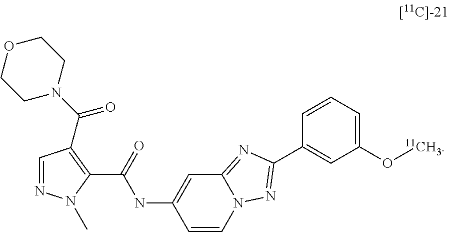

In a particular embodiment the invention relates to radiolabeled compounds of formula I for use as PDE10A PET tracers and/or autoradiography tracers.

In a particular embodiment the invention relates to radiolabeled compounds of formula I for use in a PDE10A binding study.

In a particular embodiment the invention relates to radiolabeled compounds of formula I for use in diagnostic imaging of PDE10A in the brain of a subject.

In a particular embodiment the invention relates to a method for positron emission tomography (PET) imaging of PDE10A in tissue of a subject, the method comprising:
administering an effective amount of a compound of the present invention to the subject,
allowing the compound to penetrate into the tissue of the subject; and
collecting a PET image of the CNS or brain tissue of the subject.

In a particular embodiment the invention relates to a method for the detection of PDE10A functionality in a tissue of a subject, the method comprising
administering an effective amount of a compound of the present invention to the subject,
allowing the compound to penetrate into the tissue of the subject; and
collecting a PET image of the CNS or brain tissue of the subject.

In a particular embodiment the invention relates to the use of the radiolabeled compounds of formula I for the manufacture of a composition for diagnostic imaging of PDE10A in the brain of a subject.

In a particular embodiment the invention relates to a pharmaceutical composition comprising a compound of the present invention and a pharmaceutically acceptable excipient.

### BRIEF DESCRIPTION OF THE FIGURES

FIG. 1A-1B: Specificity of autoradiography radioligand [³H]-4 binding to PDE10A: Sagittal rat brain sections were incubated with the radioligand (0.1 nM) in the absence (FIG. 1A) and presence (FIG. 1B) of 10 μM of a reference PDE10A blocker (MP-10).

FIG. 2A-2B: Specificity of autoradiography radioligand [³H]-21 binding to PDE10A: Sagittal rat brain sections were incubated with the radioligand (0.1 nM) in the absence (FIG. 2A) and presence (FIG. 2B) of 10 μM of a reference PDE10A blocker (MP-10).

FIG. 3A-3B-3C: Coronal (FIG. 3A), sagittal (FIG. 3B) and transverse (FIG. 3C) PET images in macaque brain summed from 60 to 90 min p.i. of PET tracer [¹⁸F]-20. Slices are displayed at the level of the striatum in coronal and transverse orientations, from left to right.

FIG. 4A-4B-4C: Coronal (FIG. 4A), sagittal (FIG. 4B) and transverse (FIG. 4C) PET images in macaque brain summed from 60 to 90 min p.i. of PET tracer [¹⁸F]-4. Slices are displayed at the level of the striatum in coronal and transverse orientations, from left to right.

FIG. 5: Transverse PET image of PET tracer [¹¹C]-21 in baboon brain summed from 10 to 90 min p.i.

FIG. 6: Transverse PET image of PET tracer [¹¹C]-4 in baboon brain summed from 10 to 90 min p.i.

### DEFINITIONS

The term “alkoxy” denotes a group of the formula —O—R′, wherein R′ is an alkyl group. Examples of alkoxy moieties include methoxy, ethoxy, isopropoxy, and tert-butoxy.

The term “haloalkoxy” denotes an alkoxy group wherein at least one of the hydrogen atoms of the alkoxy group has been replaced by same or different halogen atoms, particularly fluoro atoms. Examples of haloalkoxyl include monofluoro-, difluoro- or trifluoro-methoxy, -ethoxy or -propoxy, for example 3,3,3-trifluoropropoxy, 2-fluoroethoxy, 2,2,2-trifluoroethoxy, fluoromethoxy, or trifluoromethoxy. The term “perhaloalkoxy” denotes an alkoxy group where all hydrogen atoms of the alkoxy group have been replaced by the same or different halogen atoms.

The term “haloalkyl” denotes an alkyl group wherein at least one of the hydrogen atoms of the alkyl group has been replaced by same or different halogen atoms, particularly fluoro atoms. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, or trifluoromethyl. The term “perhaloalkyl” denotes an alkyl group where all hydrogen atoms of the alkyl group have been replaced by the same or different halogen atoms.

The term “alkyl” denotes a monovalent linear or branched saturated hydrocarbon group of 1 to 12 carbon atoms. In particular embodiments, alkyl has 1 to 7 carbon atoms, and in more particular embodiments 1 to 4 carbon atoms. Examples of alkyl include methyl, ethyl, propyl, isopropyl, n-butyl, iso-butyl, sec-butyl, or tert-butyl.

The term “heterocycloalkyl” denotes a monovalent saturated or partly unsaturated mono- or bicyclic ring system of 3 to 9 ring atoms, comprising 1, 2, or 3 ring heteroatoms selected from N, O and S, the remaining ring atoms being carbon. In particular embodiments, heterocycloalkyl is a monovalent saturated monocyclic ring system of 4 to 7 ring atoms, comprising 1, 2, or 3 ring heteroatoms selected from N, O and S, the remaining ring atoms being carbon. Examples for monocyclic saturated heterocycloalkyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl, or dihydropyranyl.

The term “half maximal inhibitory concentration” (IC50) denotes the concentration of a particular compound required for obtaining 50% inhibition of a biological process in vitro. IC50 values can be converted logarithmically to pIC50 values (-log IC50), in which higher values indicate exponentially greater potency. The IC50 value is not an absolute value but depends on experimental conditions e.g. concentrations employed. The IC50 value can be converted to an absolute inhibition constant (Ki) using the Cheng-Prusoff equation (Biochem. Pharmacol. (1973) 22:3099).

The term “subject” denotes a vertebrate. In certain embodiments, the vertebrate is a mammal. Mammals include humans, non-human primates such as chimpanzees and other apes and monkey species, farm animals such as cattle, horses, sheep, goats, and swine, domestic animals such as rabbits, dogs, and cats, laboratory animals including rodents, such as rats, mice, and guinea pigs. In certain embodiments, a mammal is a human. The term subject does not denote a particular age or sex.

The term “pharmaceutically acceptable salts” denotes salts which are not biologically or otherwise undesirable. Pharmaceutically acceptable salts include both acid and base addition salts.

The terms “pharmaceutically acceptable excipient” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

Pharmaceutical Compositions

Another embodiment provides pharmaceutical compositions containing the compounds of the invention and a therapeutically inert carrier, diluent or excipient, as well as methods of using the compounds of the invention to prepare such compositions and medicaments. In one example, compounds of formula [I] may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula [I] is formulated in an acetate buffer, at pH 5. In another embodiment, the compounds of formula [I] are sterile. The compound may be stored, for example, as a solid or amorphous composition, as a lyophilized formulation or as an aqueous solution.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

### EXAMPLES

Cold reference compounds (4, 20, 21) were prepared as described in WO2012076430.

List of Abbreviations:

A=non-decay corrected activity; DCM=dichloromethane; DIPEA=diisopropylethylamine; DMF=dimethylformamide; EOB=end of bombardment; EOS=end of synthesis; EtOAc=ethyl acetate; EtOH=ethanol; HATU=(0-(7-azabenzotriazol-1-yl)-N,N,N′,N′-tetramethyluronium hexafluorophosphate); Hept=Heptane; HPLC=high pressure liquid chromatography; LC-MS=liquid chromatography/mass spectrometry; MeCN=acetonitrile; K2.2.2=4,7,13,16,21,24-Hexaoxa-1,10-diazabicyclo[8.8.8]-hexacosane; NMP=N-methylpyrrolidone QMA=quaternary methylated ammonium; R1=reactor 1; R2=reactor 2; RBF=round-bottom flask; Rf=frontal ratio; RCP=Radiochemical purity; Rt=retention time; RT=room temperature; SA=Specific radioactivity; SPE=solid phase extraction; TEA=triethylamine; TFA=trifluoroacetic acid; THF=tetrahydrofuran; TLC=thin layer chromatography; Tot=toluene.

### Example 1

[¹⁸F]-2-Methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide] ([¹⁸F]-4)

General Considerations for [¹⁸F]-Radiofluorinations

HPLC quality water was used for all procedures requiring water. Solvents and reagents were purchased from Sigma Aldrich Singapore at the HPLC and highest purity grade respectively. A Synthra RN plus synthesis module (Synthra GmbH) was used for all radiochemistry procedures. The [¹⁸F]-fluoride in solution in enriched [¹⁸O]-water (98%, 2.5 mL) was produced with a PETtrace cyclotron from GE Healthcare. After loading the activity on a QMA cartridge (Waters), the fluorine-18 was transferred into a 5-mL glassy carbon reactor 1 (R1) by washing the cartridge with a solution containing MeCN (0.7 mL), K2.2.2 (12 to 15 mg) and K₂CO₃ (4 mg) in water (0.3 mL). For SPE formulation, Empore cartridge standard density (6 mL) was used. The cartridge was successively washed with EtOH (5 mL) and water (10 mL) for activation. The semi-preparative HPLC column was equilibrated with the given eluent (total volume 200 mL) before the purification of the crude.

Quality control was performed on a Perkin Elmer HPLC series 200 or Agilent 1260 series in line with a flow-RAM 1″ NaI/PMT radiodetector (LabLogic). A Phenomenex column Luna C18(2) 3 μm 100 Å 150×4.6 mm in line with a security guard cartridge was used for radio-HPLC quality control of each batch. Radio-TLC was performed with a Bioscan AR-2000 equipped with Argon/Methane gas (90/10).

### Example 1.1

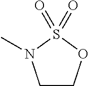

### 3-Methyl-[1,2,3]oxathiazolidine 2,2-dioxide (2)

[1,2,3]Oxathiazolidine 2,2-dioxide (1, 60 mg, 487 μM, 1.0 eq.; CAS Nr. 19044-42-9; European Journal of Medicinal Chemistry 2007, 42, 1176-1183) was combined with Tol (1.2 ml) and MeOH (0.6 ml) to give a colorless solution. After cooling down to 0° C., (diazomethyl)trimethylsilane (2 M solution in hexane, 609 μl, 1.22 mmol, 2.5 eq.) was added dropwise, and the yellow reaction mixture was stirred 30 min at 0° C. Stirring was then continued at RT overnight. The solvents were evaporated, H₂O was added and the product was extracted with EtOAc. The crude product was obtained as yellow oil (53 mg, 64%) and used as this in the next step. MS: m/z=138.1 ([M+H]⁺).

### Example 1.2

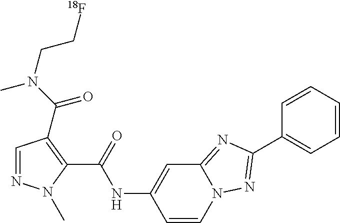 [SMILES: CN(CC[18F])C(=O)c1cnn(C)c1C(=O)Nc1ccn2nc(-c3ccccc3)nc2c1]

### [¹⁸F]-2-Methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide] ([¹⁸F]-4)

The [¹⁸F]-KF/K2.2.2 complex (52.7 GBq) was dried by azeotropic distillation under vacuum. A second distillation with MeCN (1 mL) was performed to ensure complete drying of the [¹⁸F]-KF/K2.2.2 complex. Then a solution of 3-methyl-[1,2,3]oxathiazolidine 2,2-dioxide (2) (4.2 mg, 30.6 μmol) in anhydrous MeCN (1 mL) was added to the reactive [¹⁸F]-fluoride. The resulting solution was heated at 110° C. for 10 min, then cooled down to 60° C. Under vacuum and a stream of nitrogen, MeCN was evaporated at 60° C. for 4 minutes, then at 98° C. for 3 minutes until the maximum vacuum value was reached (5 mBar without nitrogen stream, A=40.2 GBq, yield EOB=95%). After addition of TFA (500 μL) to R1 the resulting solution was heated at 110° C. for 10 minutes. The solvent was evaporated at 100° C. under vacuum and a stream of nitrogen until the maximum vacuum value was reached (5 mBar without nitrogen stream, 10 minutes). After cooling down at 25° C., DIPEA (0.2 mL) in solution in THF (1 mL) was added to R1 (sealed), the resulting solution was stirred at 25° C. for 3 minutes and transferred into R2 preloaded with 1-methyl-5-(2-phenyl-[1,2,4]triazolo[1,5-a]pyridin-7-ylcarbamoyl)-1H-pyrazole-4-carbonyl chloride (5) (CAS Nr. 1380331-83-8; WO2012076430) (3.2 mg, 8.3 mol), (A=12.7 GBq, yield EOB=38%). The solution was heated at 87° C. for 10 min and then cooled down at 60° C. before being flushed under a stream of nitrogen. The crude mixture was concentrated under vacuum for 5 min and diluted with HPLC eluent (1.8 mL; Eluent A: MeCN/AMF buffer 100 mM pH=4.0 70/30; Eluent B: MeCN/AMF buffer 100 mM pH=4.0 30/70). The resulting solution was stirred for 3 min at RT (A=5.7 GBq, yield EOB=22%). The crude mixture was loaded in the semi-preparative column and purified with a gradient elution (6.5 mL·min⁻¹: 0 to 12 min A/B 25/75 then 12 min to 25 min A/B 30/70; Column: Phenomex Luna C18(2) 100 Å 5 μm 250×10 mm; Injection volume: 2 mL; UV-detection wavelength=254 nm). The radioactive peak was collected (Rt=14.58 min, v=4.3 mL) into a round-bottom-flask containing water (45 mL). The radioactive compound was extracted by solid phase extraction at 2 mL·min⁻¹. After washing out with water (10 mL), the product was eluted off the cartridge using successively EtOH (0.5 mL) and saline (3 mL). These fractions were combined into a sterile vial containing saline (2 mL) (A=560 MBq, yield EOB=3.4%, SA EOS=327 MBq. μg⁻¹, synthesis time=183 min). The radioactive dose was quality controlled by radio-TLC (Rf=0.45 Hept/EtOAc 3/23, 100%) and analytical radio-HPLC (Rt=11.15 min, RCP=100%; HPLC system: Agilent 1260 series; Eluent A: MeCN/Water 70/30 PIC® B7; Eluent B: MeCN/Water 30/70 PIC® B7; Elution method: Isocratic at 0.63 mL·min⁻¹A/B 30/70; Column: Phenomex Luna C18(2) 100 Å 3 μm 150×4.6 mm; Injection volume: 20 μL; UV-detection wavelength=254 nm). The identity of the tracer was confirmed by spiking with cold 2-methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide] (4) as the reference compound (UV-trace: Rt=11.12 min, [¹⁸F]-trace: Rt=11.15 min

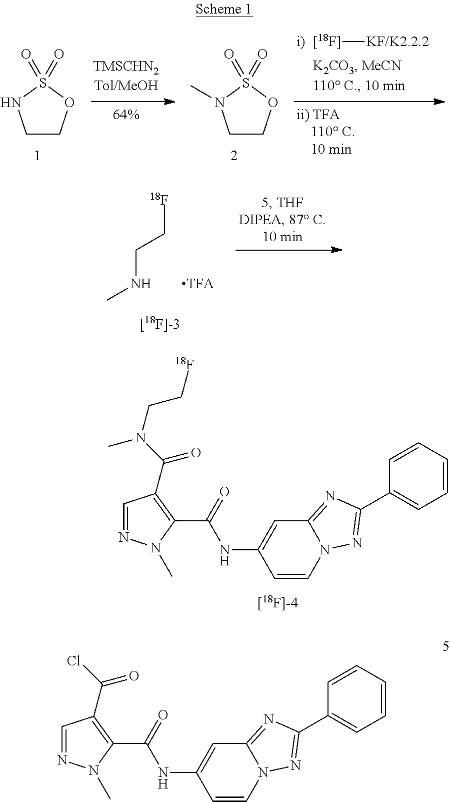

### Example 2

### [¹¹C]-2-Methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide]

Preparation of Intermediate 8

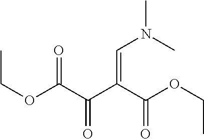 [SMILES: CCOC(=O)C(=O)/C(=C/N(C)C)C(=O)OCC]

### 2-[1-Dimethylamino-meth-(Z)-ylidene]-3-oxo-succinic acid diethyl ester (8)

To a solution of ethyl chloro-oxo-acetic acid ethyl ester (6) (10.0 g, 73.3 mmol) in DCM (50 ml) at 0° C. was added drop wise a solution of ethyl 3-(N,N-dimethylamino) acrylate (7) (10.4 g, 73.3 mmol) and pyridine in DCM (60 ml). The reaction mixture was stirred at 25° C. for 20 h. The mixture was diluted with DCM (200 ml), washed with water (2×200 ml). The aqueous layer was re-extracted with DCM (2×200 ml). The combined organic layers were washed with brine (75 ml), dried over anhydrous Na₂SO₄, filtered and evaporated off in vacuo to give 2-[1-dimethylamino-meth-(Z)-ylidene]-3-oxo-succinic acid diethyl ester (8) (15.0 g; crude, 84%) as yellow solid. LC-MS: 244.2 ([M+H]⁺).

Preparation of Intermediate 9

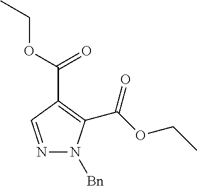 [SMILES: CCOC(=O)c1cnn(Cc2ccccc2)c1C(=O)OCC]

### 2-Benzyl-2H-pyrazole-3,4-dicarboxylic acid diethyl ester (9)

To a solution of 2-[1-dimethylamino-meth-(Z)-ylidene]-3-oxo-succinic acid diethyl ester (8) (5.00 g; 20.6 mmol; crude) in EtOH (50 ml) was added phenyl hydrazine hydrochloride (6.02 g, 30.9 mmol) at 25° C. followed by a catalytic amount of HCl_{aq} (0.2 ml) and stirring was continued for 12 h at 25° C. The solvent was removed in vacuo. The resultant crude residue was dissolved in water (50 ml) and the aqueous layer was extracted with EtOAc (2×50 ml). The combined organic layers were washed with brine (50 ml), dried over anhydrous Na₂SO₄, filtered and concentrated in vacuo. The crude material thus obtained was purified by column chromatography over normal silica gel (30% EtOAC/hexane) to afford 2-benzyl-2H-pyrazole-3,4-dicarboxylic acid diethyl ester (9) (3.5 g, 52%) as yellowish liquid. LC-MS: 303.1 ([M+H]⁺).

Preparation of Intermediate 10

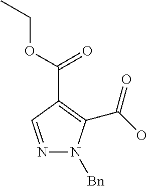 [SMILES: CCOC(=O)c1cnn(Cc2ccccc2)c1C(=O)O]

### 2-Benzyl-2H-pyrazole-3,4-dicarboxylic acid 4-ethyl ester (10)

To a solution of 2-benzyl-2H-pyrazole-3,4-dicarboxylic acid diethyl ester (9) (1.50 g, 5.00 mmol) in a mixture of THF (15 ml), MeOH (7 ml) and water (7 ml) was added lithium hydroxide monohydrate (208 mg, 5.00 mmol) at 0° C. The reaction mixture was stirred at 25° C. for 1 h. The solvents were removed in vacuo. The resultant crude material was diluted with water (15 ml). The aqueous layer was washed with EtOAc (2×10 ml), cooled to 0° C., and acidified (pH 5) with an aqueous 1N HCl solution. The resultant precipitated solid was filtered and dried under vacuum to give 2-benzyl-2H-pyrazole-3,4-dicarboxylic acid 4-ethyl ester (10) as white solid. (0.7 g, 51%) LC-MS: 275.1 ([M+H]⁺).

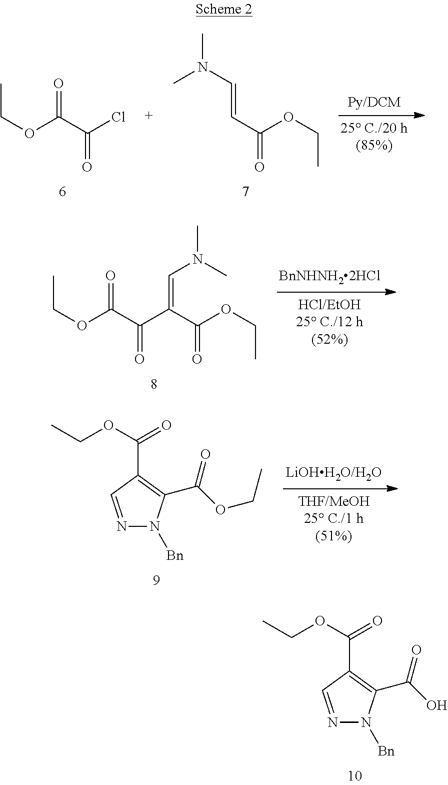

### Example 2.1

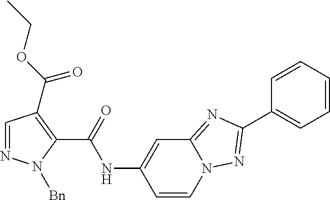 [SMILES: CCOC(=O)c1cnn(Cc2ccccc2)c1C(=O)Nc1ccn2nc(-c3ccccc3)nc2c1]

### 1-Benzyl-5-(2-phenyl-[1,2,4]triazolo[1,5-a]pyridin-7-ylcarbamoyl)-1H-pyrazole-4-carboxylic acid ethyl ester (12)

A solution of 2-benzyl-2H-pyrazole-3,4-dicarboxylic acid 4-ethyl ester (intermediate 10) (0.50 g, 1.80 mmol) in oxalyl chloride (10 ml) was heated for 2 h at 50° C. Volatilities were removed carefully in vacuo. To the crude acid chloride was then added pyridine (15 ml) drop wise at 0° C. followed by 2-phenyl-[1,2,4]triazolo[1,5-a]pyridin-7-ylamine (11, CAS Nr. 1380331-14-5, WO2012076430) (450 mg, 1.80 mmol). The resultant reaction mixture was stirred for 10 min at 25° C. The mixture was poured onto ice cold water. The resultant solid thus obtained was filtered, washed sequentially with water and hexane and finally dried by azeotroping with Tol to yield 1-benzyl-5-(2-phenyl-[1,2,4]triazolo[1,5-a]pyridin-7-ylcarbamoyl)-1H-pyrazole-4-carboxylic acid ethyl ester (12) (0.45 g, 53%) as off-white solid. LC-MS: 467.0 ([M+H]⁺).

### Example 2.2

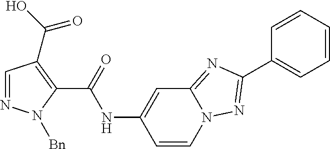 [SMILES: O=C(O)c1cnn(Cc2ccccc2)c1C(=O)Nc1ccn2nc(-c3ccccc3)nc2c1]

### 1-Benzyl-5-(2-phenyl-[1,2,4]triazolo[1,5-a]pyridin-7-ylcarbamoyl)-1H-pyrazole-4-carboxylic acid (13)

To a solution of 1-benzyl-5-(2-phenyl-[1,2,4]triazolo[1,5-a]pyridin-7-ylcarbamoyl)-1H-pyrazole-4-carboxylic acid ethyl ester (12) (0.50 g, 1.07 mmol) in THF (25 ml) at 0° C. was added an aqueous solution of lithium hydroxide monohydrate (1M; 1.34 ml, 1.34 mmol). The reaction mixture was then stirred at 25° C. for 2 h. The mixture was acidified (pH-2) with aqueous 1N HCl solution. The resultant precipitate was filtered, washed with water followed by hexane and finally dried by azeotroping with Tol to give 1-benzyl-5-(2-phenyl-[1,2,4]triazolo[1,5-a]pyridin-7-ylcarbamoyl)-1H-pyrazole-4-carboxylic acid (13) (0.25 g, 53%) as off-white solid. LC-MS: 439.1 ([M+H]⁺).

### Example 2.3

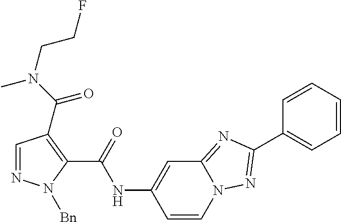 [SMILES: CN(CCF)C(=O)c1cnn(Cc2ccccc2)c1C(=O)Nc1ccn2nc(-c3ccccc3)nc2c1]

### 2-Benzyl-2H-pyrazole-3,4-dicarboxylicacid 4-[(2-fluoro-ethyl)-methyl-amide]3-[(2-phenyl-[1,2,4]triazolo[1,5-a]pyridin-7-yl)-amide] (14)

To a solution of 1-benzyl-5-(2-phenyl-[1,2,4]triazolo[1,5-a]pyridin-7-ylcarbamoyl)-1H-pyrazole-4-carboxylic acid (13) (0.30 g, 0.684 mmol) in DMF (5 ml) were added HATU (0.54 g, 1.44 mmol), (2-fluoro-ethyl)-methyl-amine hydrochloride (0.309 g, 2.74 mmol) and DIPEA (0.48 ml, 2.74 mmol) at 0° C. The resultant reaction mixture was stirred at 25° C. for 16 h. The mixture was diluted with water (10 ml) and stirred for 15 min. The resultant precipitated solid was filtered, washed thoroughly with water and dried by azeotroping with Tol followed by further drying under vacuum that yielded 2-benzyl-2H-pyrazole-3,4-dicarboxylicacid4-[(2-fluoro-ethyl)-methyl-amide]3-[(2-phenyl-[1,2,4]triazolo[1,5-a]pyridin-7-yl)-amide](14) (200 mg, 69%) as off-white solid. LC-MS: 498.0 ([M+H]⁺).

### Example 2.4

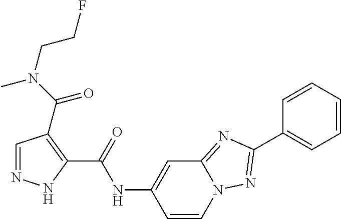 [SMILES: CN(CCF)C(=O)c1cn[nH]c1C(=O)Nc1ccn2nc(-c3ccccc3)nc2c1]

### 2H-Pyrazole-3,4-dicarboxylic acid 4-[(2-fluoro-ethyl)-methyl-amide]3-[(2-phenyl-[1,2,4]triazolo[1,5-a]pyridin-7-yl)-amide] (15)

To a solution of 2-benzyl-2H-pyrazole-3,4-dicarboxylicacid4-[(2-fluoro-ethyl)-methyl-amide]3-[(2-phenyl-[1,2,4]triazolo[1,5-a]pyridin-7-yl)-amide] (14) (1.6 g, 3.2 mmol) in anhydrous DCM (100 ml), was added boron tribromide (1M solution in DCM, 6.4 ml, 6.4 mmol) at 0° C. under nitrogen. The mixture was allowed to stir for 0.5 h at 0° C. The reaction mixture was neutralized by an aqueous 1N NaOH solution. The solvents were removed under vacuo. The crude material thus obtained was purified by column chromatography over silica gel (5% MeOH/DCM) to afford 2H-pyrazole-3,4-dicarboxylic acid 4-[(2-fluoro-ethyl)-methyl-amide]3-[(2-phenyl-[1,2,4]triazolo[1,5-a]pyridin-7-yl)-amide] (15) (500 mg, 38%) as off-white solid. LC-MS: 408.1 ([M+H]⁺).

### Example 2.5

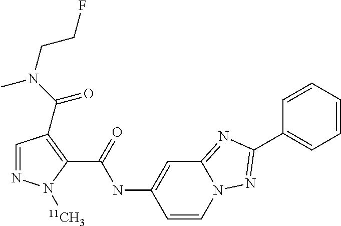 [SMILES: CN(CCF)C(=O)c1cnn([11CH3])c1C(=O)Nc1ccn2nc(-c3ccccc3)nc2c1]

### [¹¹C]-2-Methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide] ([¹¹C]-4)

To a 1 mL V-vial was added 2H-pyrazole-3,4-dicarboxylic acid 4-[(2-fluoro-ethyl)methyl-amide]3-[(2-phenyl-[1,2,4]triazolo[1,5-a]pyridin-7-yl)-amide] (15) (4 mg). The precursor was dissolved in 0.1 mL of dimethylformamide. In a separate 1 mL V-vial, 1,4,7,10,13,16-hexaoxacyclooctadecane (0.56 mg) was dissolved in 0.1 mL of dimethylformamide, after which 9.5 μL of 1M potassium tert-butoxide in tetrahydrofuran was added. The contents of the 2 vials were thoroughly mixed and the final vial was capped with a septum seal before addition of [¹¹C]methyl iodide. [¹¹C]Methyl iodide, produced from [¹¹C]carbon dioxide and carried by a stream of helium, was trapped in the above solution. Following a plateau of radioactivity, the reaction vial was left at room temperature for 2 min, and then quenched with 0.2 mL of preparative HPLC mobile phase consisting of 30% acetonitrile/70% aqueous buffer (57 mM TEA adjusted to pH 3.2 with o-phosphoric acid). The crude reaction product was purified by reverse-phase HPLC (Waters XBridge C18 10×150 mm, 10μ) at 15 mL/min at 254 nm. The radioproduct (Rt=7.7 min) that was separated from the precursor (Rt=2.2 min) was remotely collected in a reservoir of 50 mL water. The product fraction in a reservoir of water was loaded onto the C18 Sep-Pak. The C18 Sep-Pak was then flush to waste with 10 mL 0.9% Sodium Chloride Injection. The final product was eluted from the C18 Sep-Pak with 1 mL of Ethanol followed by 14 mL of 0.9% Sodium Chloride Injection, through a sterilizing 0.22μ filter in a sterile, pyrogen-free vial.

The average non-decay corrected radiochemical yield for [¹¹C]-2-methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide] ([¹¹C]-4) was approx. 10%. An aliquot (0.1 mL) was assayed for radioactivity and checked by analytical HPLC (35% acetonitrile/65% aqueous buffer (57 mM TEA adjusted to pH 3.2 with o-phosphoric acid); Waters XBridge C18 10×150 mm, 10μ) at 2 mL/min at 254 nm. A single radioactive peak (Rt=4.5 min) corresponding to 2-methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide (4) was observed. The specific radioactivity at the end-of-synthesis determined by relating radioactivity to the mass associated with the UV absorbance peak of carrier was over 9000 mCi/μmole at end of synthesis.

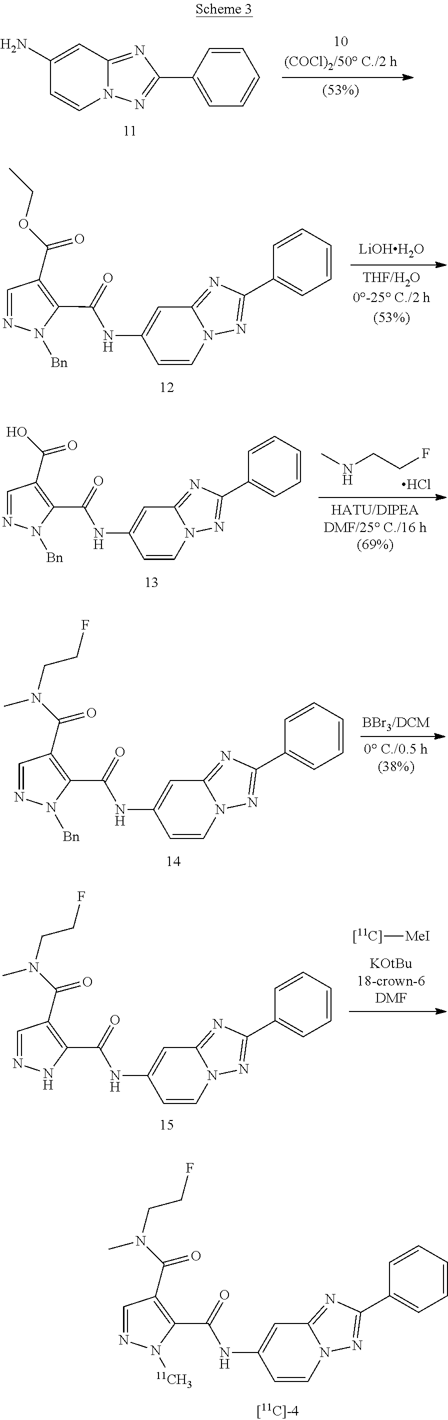

### Example 3

### [³H]-2-Methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide]

### Example 3.1

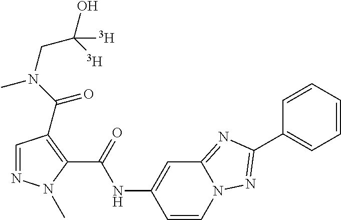 [SMILES: [3H]C([3H])(O)CN(C)C(=O)c1cnn(C)c1C(=O)Nc1ccn2nc(-c3ccccc3)nc2c1]

### [³H]-2-Methyl-2H-pyrazole-3,4-dicarboxylic acid 4-[(2-hydroxy-ethyl)-methyl-amide]3-[(2-phenyl-[1,2,4]triazolo[1,5-a]pyridin-7-yl)-amide ([³H]-17)

A solution of {methyl-[1-methyl-5-(2-phenyl-[1,2,4]triazolo[1,5-a]pyridin-7-ylcarbamoyl)-1H-pyrazole-4-carbonyl]amino}-acetic acid methyl ester (16) (CAS Nr. 1380330-71-1; WO2012076430) (22 mg, 49 μmol) in 1 ml of THF is added to a solution of 50 μmol of lithium borotritide in 150 μl of THF/heptane (2:1) at 0° C. After stirring for 3 h at room temperature the reaction mixture is quenched with 0.2 ml of acetone before a solution of 20 μl of TFA in 0.4 ml of THF is added. The solvents are removed under reduced pressure, the resulting residue is dissolved in 10 ml of THF and the solution is passed through a short silica Sep-Pak cartridge. After evaporation of the solvent the resulting crude product is purified by HPLC (Nuleodur C18 Gravity, elution with 0.1% TFA in acetonitrile/0.1% TFA in water 10:90 to 46:54 in 9 min, then 95% acetonitrile for 3 min). The product fractions are collected and subsequently neutralized by addition of aqueous bicarbonate. The pure compound is isolated by solid phase extraction (StrataX). After washing the cartridge with water the product is eluted with ethanol to yield 325 mCi of [³H]-2-methyl-2H-pyrazole-3,4-dicarboxylic acid 4-[(2-hydroxy-ethyl)-methyl-amide]3-[(2-phenyl-[1,2,4]triazolo[1,5-a]pyridin-7-yl)-amide] in 97% radiochemical purity.

### Example 3.2

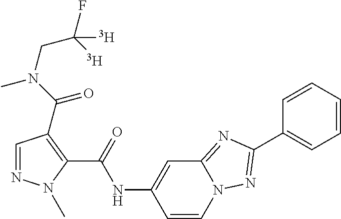 [SMILES: [3H]C([3H])(F)CN(C)C(=O)c1cnn(C)c1C(=O)Nc1ccn2nc(-c3ccccc3)nc2c1]

### [³H]-2-Methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide ([³H]-4)

To a solution of 98 mCi of [³H]-2-methyl-2H-pyrazole-3,4-dicarboxylic acid 4-[(2-hydroxy-ethyl)-methyl-amide]3-[(2-phenyl-[1,2,4]triazolo[1,5-a]pyridin-7-yl)-amide] ([³H]-17) in 100 μl of DMF are added 16 μl (92 μmol) of Huenig's base, 5 μl (31 μmol) of triethylamine trihydrofluoride, and 8.4 μl (27 μmol) of perfluorobutanesulfonyl fluoride. The reaction mixture is stirred for 67 h at room temperature. After dilution with 2.5 ml of water the crude product is pre-purified by solid phase extraction (StrataX, ethanol). After final purification by HPLC (Nuleodur C18 Gravity, elution with 0.1% TFA in acetonitrile/0.1% TFA in water 40:60 to 49:51 in 6 min) the product fractions are collected and subsequently neutralized by addition of aqueous bicarbonate. The pure compound is isolated by solid phase extraction (StrataX). After washing the cartridge with water the product is eluted with ethanol to give 21 mCi of [³H]-2-methyl-2H-pyrazole-3,4-dicarboxylic acid 4-[(2-fluoro-ethyl)-methyl-amide]3-[(2-phenyl-[1,2,4]triazolo[1,5-a]pyridin-7-yl)-amide] ([³H]-4) in >97% radiochemical purity and a specific activity of 47 Ci/mmol.

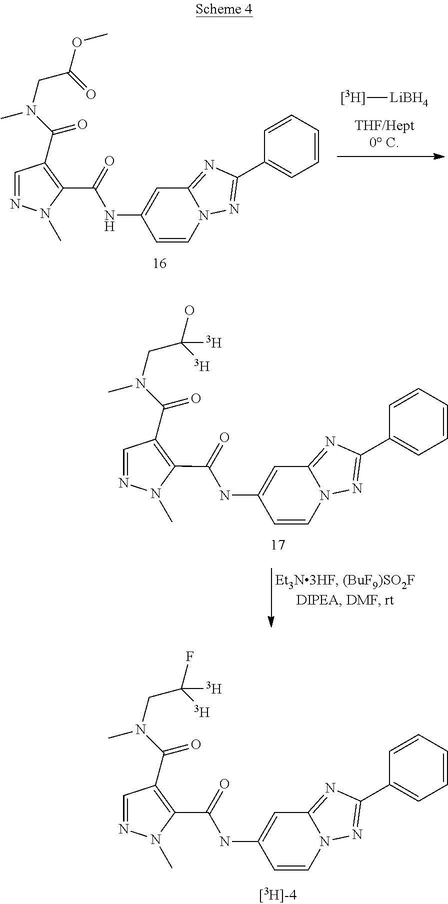

### Example 4

### [¹⁸F]-2-Methyl-4-(morpholine-4-carbonyl)-2H-pyrazole-3-carboxylic acid {2-[3-(2-fluoro-ethoxy)-phenyl]-[1,2,4]triazolo[1,5-a]pyridin-7-yl}-amide ([¹⁸F]-20)

### Example 4.1

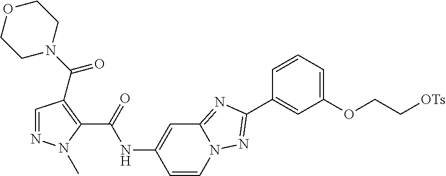 [SMILES: Cc1ccc(S(=O)(=O)OCCOc2cccc(-c3nc4cc(NC(=O)c5c(C(=O)N6CCOCC6)cnn5C)ccn4n3)c2)cc1]

### Toluene-4-sulfonic acid 2-[3-(7-{[2-methyl-4-(morpholine-4-carbonyl)-2H-pyrazole-3-carbonyl]-amino}-[1,2,4]triazolo[1,5-a]pyridin-2-yl)-phenoxy]-ethyl ester (19)

The starting material 2-methyl-4-(morpholine-4-carbonyl)-2H-pyrazole-3-carboxylic acid [2-(3-hydroxy-phenyl)-[1,2,4]triazolo[1,5-a]pyridin-7-yl]-amide (18) (CAS Nr. 1380330-59-5; WO2012076430) (100 mg, 223 μmol) was combined with NMP (2.0 ml) to give a light yellow solution. Ethane-1,2-diyl bis(4-methylbenzenesulfonate) (166 mg, 447 μmol) and cesium carbonate (73 mg, 223 μmol) were added, and the reaction mixture was stirred over night at 60° C. EtOAc and H₂O were added. The organic layer was collected and dried over Na₂SO₄. After filtration and evaporation of the solvents, the crude compound was purified by flash chromatography (10 g SiO2 cartridge, CH₂Cl₂ to CH₂Cl₂/MeOH/NH₃ aq. 300:10:1). A final HPLC purification yielded the title compound (38 mg, 26%) as off-white foam. LC-MS: 646.2 ([M+H]⁺).

### Example 4.2

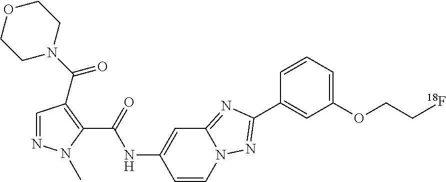 [SMILES: Cn1ncc(C(=O)N2CCOCC2)c1C(=O)Nc1ccn2nc(-c3cccc(OCC[18F])c3)nc2c1]

### [¹⁸F]-2-Methyl-4-(morpholine-4-carbonyl)-2H-pyrazole-3-carboxylic acid {2-[3-(2-fluoro-ethoxy)-phenyl]-[1,2,4]triazolo[1,5-a]pyridin-7-yl}-amide ([¹⁸F]-20)

The [¹⁸F]-KF/K2.2.2 complex (26.0 GBq) was dried by azeotropic distillation under vacuum. A second distillation with MeCN (1 mL) was performed to ensure complete drying of the [¹⁸F]-KF/K2.2.2 complex. Then a solution of toluene-4-sulfonic acid 2-[3-(7-{[2-methyl-4-(morpholine-4-carbonyl)-2H-pyrazole-3-carbonyl]-amino}-[1,2,4]triazolo[1,5-a]pyridin-2-yl)phenoxy]-ethyl ester (19) (5.8 mg, 8.98 μmol) in anhydrous DMSO (0.8 mL) was added to the reactive [¹⁸F]-fluoride. The resulting solution was heated at 120° C. for 30 minutes, then cooled down to 40° C. The crude mixture was diluted with HPLC eluent (4 mL; Eluent A: H₂O with TFA 0.02%; Eluent B: MeCN with TFA 0.02%) and stirred for 3 minutes at RT. The crude mixture was loaded in the semi-preparative HPLC column for purification (Gradient elution at 5 mL·min⁻¹: 0 to 20 min A/B 40/60 then A/B 70/30; Column: Nucleodur C18 Pyramid 110 Å 7 μm 250×10 mm; Injection volume: 5 mL; UV-detection wavelength=254 nm). The radioactive peak was collected (Rt=18.35 min, v=11 mL) into a round-bottom-flask containing water (50 mL). The radioactive compound was extracted by solid phase extraction at 5 mL·min⁻¹. After washing out with water (10 mL), the product was eluted off the cartridge using successively EtOH (1 mL) and saline (7 mL). These fractions were combined into a sterile vial containing saline (3 mL) (A=2.32 GBq, yield EOB=18%, SA EOS=101 MBq·μg⁻¹, synthesis time=107 min). The radioactive dose was quality controlled by radio-TLC (Rf=0.25 Hept/EtOAc 15/85) and analytical radio-HPLC (Rt=4.18 min, RCP=99.8%; HPLC system: Perkin Elmer HPLC series 200; Eluent A: H₂O with TFA 0.02%; Eluent B: MeCN with TFA 0.02%; Elution method: Isocratic A/B 40/60 at 1.5 mL·min⁻¹; Column: Thermo Scientific Hypersil Gold C18 175 Å 3 μm 150×4.6 mm; Injection volume: 20 μL; UV-detection wavelength=254 nm). The identity of the tracer was confirmed by spiking with cold 2-methyl-4-(morpholine-4-carbonyl)-2H-pyrazole-3-carboxylic acid {2-[3-(2-fluoro-ethoxy)-phenyl]-[1,2,4]triazolo[1,5-a]pyridin-7-yl}-amide as the reference compound (UV-trace: Rt=4.03 min, [¹⁸F]-trace: Rt=4.14 min).

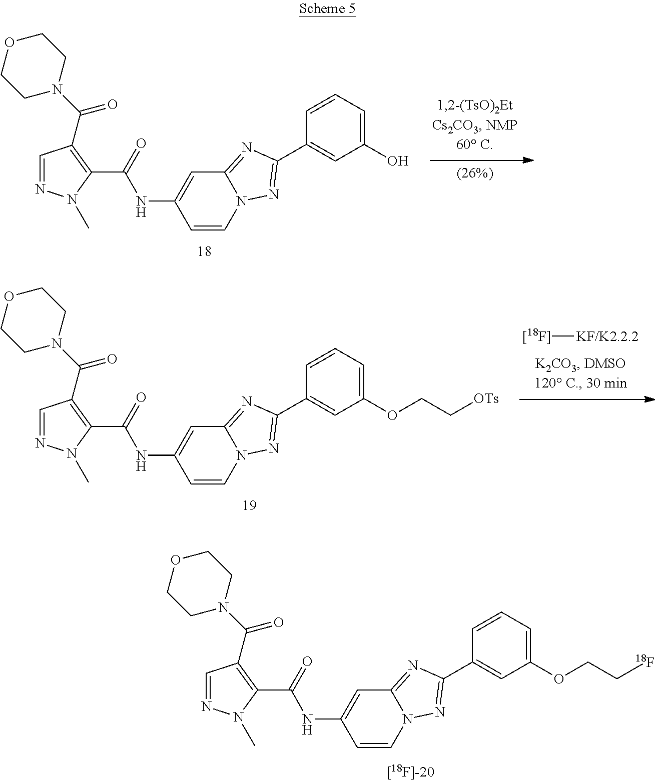

### Example 5

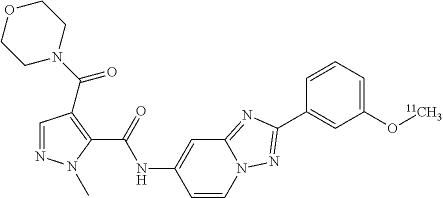 [SMILES: Cn1ncc(C(=O)N2CCOCC2)c1C(=O)Nc1ccn2nc(-c3cccc(O[11CH3])c3)nc2c1]

### [¹¹C]-2-Methyl-4-(morpholine-4-carbonyl)-2H-pyrazole-3-carboxylic acid [2-(3-methoxy-phenyl)-[1,2,4]triazolo[1,5-a]pyridin-7-yl]-amide ([¹¹C]-21)

To a 1 mL V-vial was added precursor 2-methyl-4-(morpholine-4-carbonyl)-2H-pyrazole-3-carboxylic acid [2-(3-hydroxy-phenyl)-[1,2,4]triazolo[1,5-a]pyridin-7-yl]-amide (18) (CAS Nr. 1380330-59-5; WO2012076430). The precursor was dissolved in 0.2 mL of dimethylsulfoxide. Three microliters of 5 N sodium hydroxide was added, and the vial was capped with a septum seal before addition of [¹¹C]-methyl iodide. [¹¹C]-Methyl iodide, produced from [¹¹C]-carbon dioxide and carried by a stream of helium, was trapped in the above solution. Following a plateau of radioactivity, the reaction vial was heated in an 80° C. water bath for 3 min, and then quenched with 0.2 mL of preparative HPLC mobile phase consisting of 30% acetonitrile/70% aqueous buffer (57 mM TEA adjusted to pH 3.2 with o-phosphoric acid). The crude reaction product was purified by reverse-phase HPLC (Waters XBridge C18 10×150 mm, 10μ) at 10 mL/min at 254 nm. The radioproduct (Rt=9.5 min) that was separated from the precursor (Rt=3.2 min) and was remotely collected in a reservoir of 50 mL water. The product fraction in a reservoir of water was loaded onto the C18 Sep-Pak. The C18 Sep-Pak is then flush to waste with 10 mL 0.9% Sodium Chloride Injection. The product [¹¹C]-21 was eluted from the C18 Sep-Pak with 1 mL of Ethanol followed by 14 mL of 0.9% Sodium Chloride Injection, through a sterilizing 0.22μ filter in a sterile, pyrogen-free vial.

The average non-decay corrected radiochemical yield for [¹¹C]-21 was approx. 25%. An aliquot (0.1 mL) was assayed for radioactivity and checked by analytical HPLC (40% acetonitrile/60% aqueous buffer (57 mM TEA adjusted to pH 3.2 with o-phosphoric acid; Waters XBridge C18 10×150 mm, 10μ) at 2 mL/min at 254 nm. A single radioactive peak (Rt=2.3 min) corresponding to [¹¹C]-2-methyl-4-(morpholine-4-carbonyl)-2H-pyrazole-3-carboxylic acid [2-(3-methoxy-phenyl)-[1,2,4]triazolo[1,5-a]pyridin-7-yl]-amide ([¹¹C]-21) was observed. The specific radioactivity at the end-of-synthesis determined by relating radioactivity to the mass associated with the UV absorbance peak of carrier was over 7500 mCi/μmole at end of synthesis.

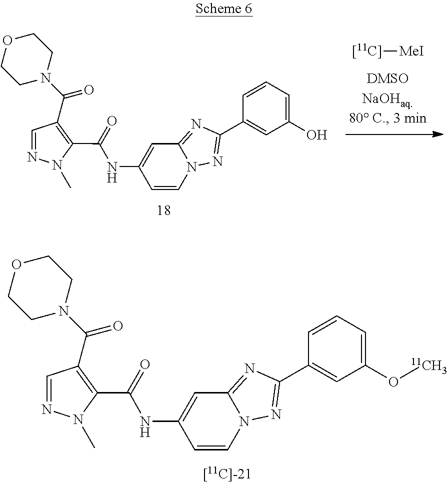

### Example 6

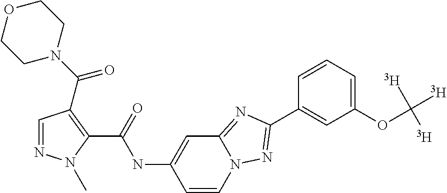 [SMILES: [3H]C([3H])([3H])Oc1cccc(-c2nc3cc(NC(=O)c4c(C(=O)N5CCOCC5)cnn4C)ccn3n2)c1]

### [³H]-2-Methyl-4-(morpholine-4-carbonyl)-2H-pyrazole-3-carboxylic acid [2-(3-methoxy-phenyl)-[1,2,4]triazolo[1,5-a]pyridin-7-yl]-amide ([³H]-21)

A solution of 0.67 mg (1.5 μmol) of the phenol precursor (18) in 300 μl of DMF is added to a solution of 50 mCi (0.69 μmol) of [³H]methyl nosylate (methyl 4-nitrobenzene sulfonate[methyl-³H]) in 200 μl of DMF followed by the addition of 0.7 mg (3.6 μmol) of cesium carbonate. After stirring for 3 h at room temperature the reaction mixture is diluted with aqueous ammonium chloride and extracted with tert-butyl methyl ether. After separation and evaporation of the organic layer the resulting crude product is purified by HPLC (XBridge C18, elution with acetonitrile/water 20:80 to 70:30 in 15 min). The product fractions are collected, subsequently the pure compound is isolated by solid phase extraction (Sep-Pak C18). After elution with ethanol 5 mCi of [³H]-2-methyl-4-(morpholine-4-carbonyl)-2H-pyrazole-3-carboxylic acid [2-(3-methoxy-phenyl)-[1,2,4]triazolo[1,5-a]pyridin-7-yl]-amide ([³H]-21) are obtained in >99.4% radiochemical purity and a specific activity of 76 Ci/mmol.

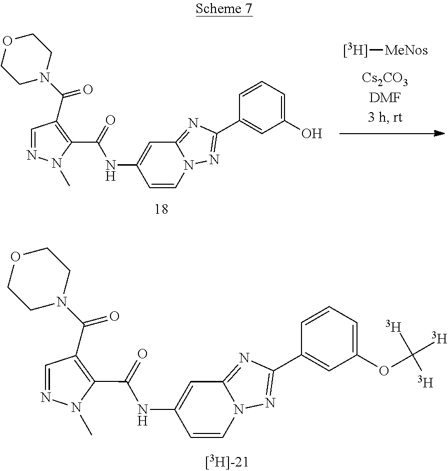

Pharmacological Tests

The following test was carried out in order to determine the activity of the compounds of the present invention. PDE10A activity of the compounds of the present invention was determined using a Scintillation Proximity Assay (SPA)-based method similar to the one previously described (Fawcett, L. et al., Proc. Natl. Acad. Sci. USA 2000, 97(7), 3702-3707).

The human PDE10A full length assay was performed in 96-well micro titer plates. The reaction mixture of 50 μl contained 20 mM HEPES pH=7.5/10 mM MgCl₂/0.05 mg/ml BSA (Sigma cat. # A-7906), 50 nM cGMP (Sigma, cat. # G6129) and 50 nM [³H]-cGMP (GE Healthcare, cat. # TRK392 S.A. 13.2 Ci/mmol), 3.75 ng/well PDE10A enzyme (Enzo Life Science, Lausen, Switzerland cat # SE-534) with or without a specific test compound. A range of concentrations of the potential inhibitor was used to generate data for calculating the concentration of inhibitor resulting in 50% of the effect (e.g. IC₅₀, the concentration of the competitor inhibiting PDE10A activity by 50%). Non-specific activity was tested without the enzyme. The reaction was initiated by the addition of the substrate solution (cGMP and [³H]-cGMP) and allowed to progress for 20 minutes at room temperature. The reaction was terminated by adding 25 μl of YSi-SPA scintillation beads (GE Healthcare, cat. # RPNQ0150) in 18 mM zinc sulphate solution (stop reagent). After 1 h under shaking, the plate was centrifuged one minute at 170 g to allow beads to settle. Afterwards, radioactive counts were measured on a Perkin Elmer TopCount Scintillation plate reader.

The compounds according to formula (I) have an IC₅₀ value below 10 μM, more specifically below 5 μM, yet more specifically below 1 μM. The following table shows data for the tracers.

| | PDE10A inhibition |
|---|---|
| Example | IC₅₀ [nM] |
| 4 | 0.94 |
| 20 | 0.59 |
| 21 | 5.1 |

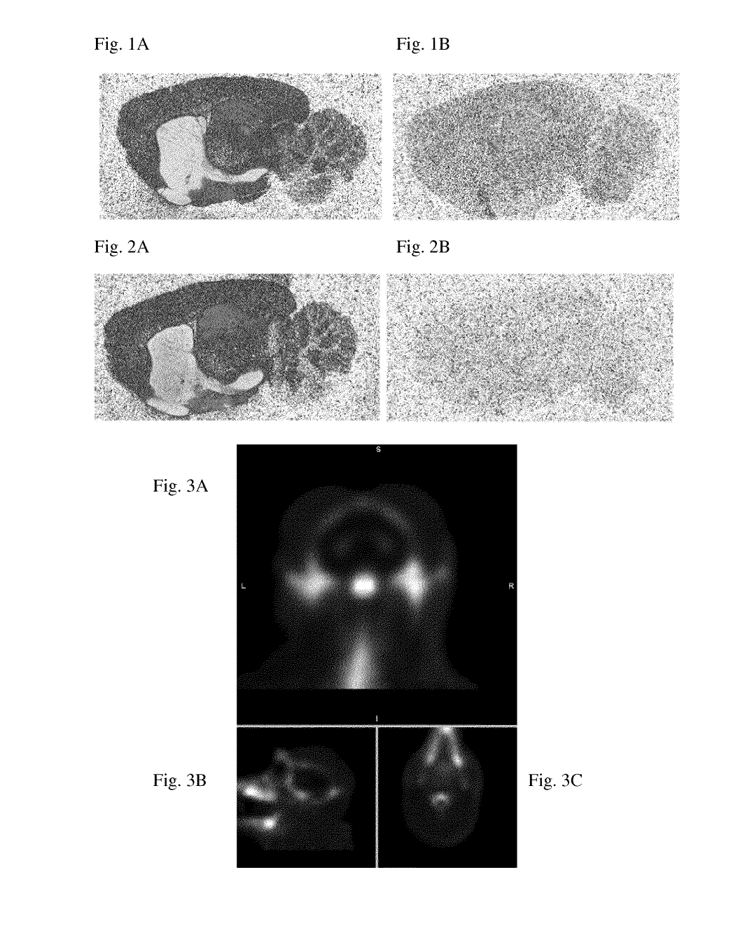

In Vitro Autoradiography (FIGS. 1 and 2)
1) In Vitro Autoradiography

The distribution of tritiated radioligand binding sites as well as the binding specificity for PDE10A was investigated by in vitro autoradiography using male Sprague-Dawley rats. Animals were sacrificed and their brains were rapidly removed and frozen in dry ice powder. Ten μm-thick sagittal sections were cut in a Cryostat microtome and thaw-mounted on adhesion glass slides. Brain sections were first incubated for 10 min in Ringer buffer (NaCl 120 mM, KCl 5 mM, CaCl₂ 2 mM, MgCl₂ 1 mM, Tris-HCl 50 mM pH 7.4) at RT and then for 60 min in Ringer buffer containing the radioligand. For the evaluation of non-specific-binding (NSB) of the radiotracer an additional series of sections was incubated with Ringer buffer containing the radiotracer and a reference high affinity PDE10A inhibitor (MP-10).

At the end of the incubation, sections were rinsed 3×5 min in ice-cold Ringer buffer and then rapidly dipped once in distilled water at 4° C. Slide-mounted brain sections were dried under a flow of cold air for 3 h and exposed together with [³H]-microscale to a Fuji Imaging plate for 5 days. The imaging plate was then scanned in a high resolution Fuji BAS reader. The total amount of radiotracer bound to the brain areas of interest (TB) was measured using the MCID image analysis program and expressed as fmol of bound radiotracer/mg of protein. The amount of radioligand specifically bound to PDE10A (% SB) was calculated according to the formula % SB=100-(NSB/TB*100). The results obtained showed that specific binding amounted to more than 95% (see FIGS. 1 and 2).

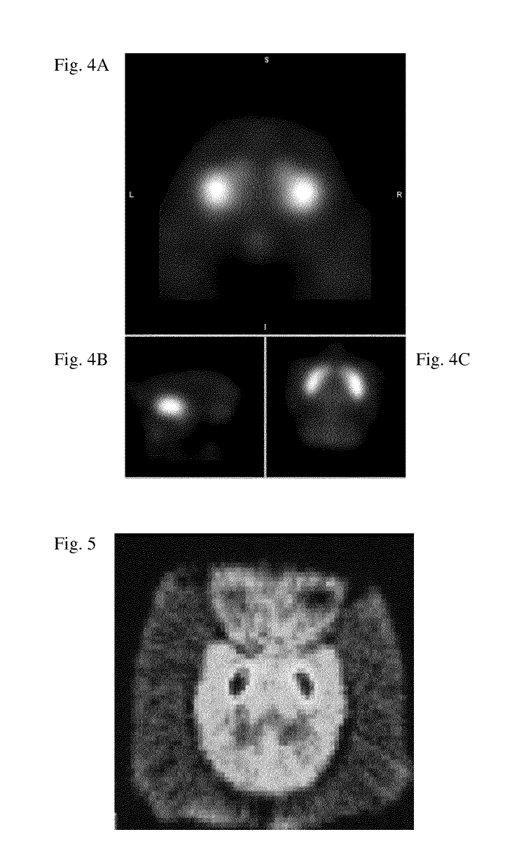

PET Imaging Experiments in Cynomolgus Monkey (FIGS. 3 and 4)

All doses of fluorine-18 labelled radiotracers are infused i.v. over 1 minute maximum under isoflurane anaesthesia; after administration the cannula is flushed with 5 mL of saline. Radiotracers are formulated in sterile saline containing max 10% EtOH.

Animals, male cynomolgus monkey (Macaca fascicularis of Mauritian origin) with an average body-weight of 7-8 kg are fasted overnight or for a minimum of 6 hours prior to scanning Animal preparation is performed under no anesthesia. The animal is chaired and the arms/legs restrained. An intravenous catheter is inserted in the right and left cephalic vein for the injection of the radiotracer and as access point for the fluid drip. Prior to scanning the animal is anesthetized with propofol (3-6 mg/kg), transferred to the PET camera bed where an endotracheal tube is inserted for administration of gaseous anesthesia. Monkeys are positioned supine in a PET compatible pediatric restraint on the scanner couch, and placed under isoflurane anesthesia (1-2%). The endotracheal tube is connected to a capnograph for monitoring respiratory rate and endtidal carbon dioxide levels. The percent arterial oxygen saturation is monitored continuously via pulse oximetry. Under aseptical conditions the femoral artery is cannulated for arterial blood sampling. The animal is positioned in the bore of the PET scanner utilizing CT scout scans. Once correct position is confirmed a transmission scan is acquired.

The animal is then dosed with the radiotracer. Dynamic three-dimensional (3D) PET scans are performed on a General Electric Discovery VCT whole body scanner; 35 simultaneous slices, axial field of view 15.7 cm. Dynamic emission data are acquired for 180 minutes at a bed position with the head in the center of the field of view. The exact scan time is recorded along with the time of administration of the radiotracer and the blood sampling time points.

During each acquisition, arterial blood samples are collected from the femoral artery for the determination of the whole blood and plasma input functions. The radioactivity concentrations in whole blood and in plasma are measured at 10 s, 20 s, 30 s, 40 s, 50 s, 60 s, 90 s, 2 min 5 min, 10 min, 20 min, 40 min, 60 min, 90 min, 120 min and 180 min post administration of the radiotracer using a dedicated PET gamma counter (Wizard2, Perkin Elmer) calibrated and normalized to measure 18F. Whole blood samples are weighed and counted. The plasma samples are handled in the same manner.

The fraction of radioactivity in plasma corresponding to authentic radiotracer is determined by radio-HPLC of arterial plasma samples collected at 2, 5, 10, 20, 40, 60, 90 and 120 min after administration.

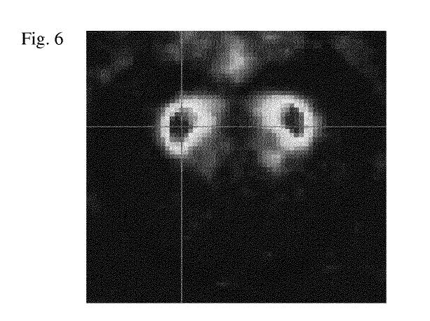

In Vivo PET Imaging in the Baboon (FIGS. 5 and 6)

The PET experiments were carried out in male baboons (papio anubis). Animals were fasted for 12 hours prior to the PET study. Baboons were initially sedated intramuscularly with ketamine hydrochloride with restraint dosages of 5-7 mg/kg to achieve a superficial level of anesthesia and then maintained on continuous propofol intravenous infusion at 0.3-0.4 mg/kg/h (DIPRIVAN® Injectable Emulsion). Circulatory volume was maintained by infusion of isotonic saline. A femoral arterial catheter was inserted for blood sampling. Physiological vital signs including heart rate, ECG, blood pressure and oxygen saturation were continuously monitored throughout the study. The animal was positioned in an ECAT HRRT® brain PET scanner (High Resolution Research Tomograph, CPS Innovations, Inc., Knoxville, Tenn.). The head of the animal was fitted with a thermoplastic mask that was attached to a head holder for reproducible fixation. A 6 min transmission scan with a 1 mCi Cs-137 point source was initially done for attenuation correction. The [11C]-radiotracer (approximately 20 mCi or 1.5 μg) was administered intravenously as a 1 minute bolus injection. PET scanning and arterial blood sampling was initiated upon start of the radiotracer administration and PET images were acquired from 0 to 120 minutes following administration of the radiotracer. Emission PET scans were reconstructed using the iterative ordered-subset expectation-maximization (OSEM) algorithm correcting for attenuation, scatter and dead-time. A standard VOI template was transferred to each individual animal's baseline PET. The results of the PET imaging studies showed that the radiotracer readily penetrated in the baboon brain and accumulated specifically in PDE10A expressing brain region such as the caudate putamen.

1. A radiolabeled compound of formula I 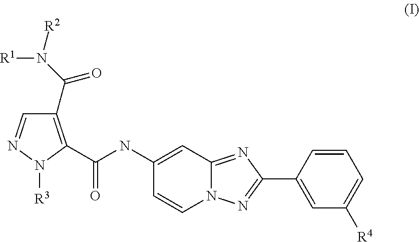
wherein
R¹ and R² are independently selected from C₁₋₇ alkyl, C₁₋₇ haloalkyl, R¹ and R², together with the nitrogen atom to which they are attached, form heterocycloalkyl,
R³ is C₁₋₇ alkyl,
R⁴ is hydrogen, C₁₋₇ alkoxy or C₁₋₇ haloalkoxy and
wherein either R¹, R², R³ or R⁴ is labeled with a radionuclide selected from ³H, ¹¹C and ¹⁸F.

2. The radiolabeled compound of claim 1, wherein
R¹ is C₁₋₇ alkyl,
R² is C₁₋₇ fluoroalkyl,
R³ is methyl,
R⁴ is hydrogen, wherein either R² is labeled with ¹⁸F or ³H, or R³ is labeled with ¹¹C.

3. The radiolabeled compound of claim 1, wherein
R¹ and R² together with the nitrogen atom to which they are attached, form a heterocycloalkyl, preferably morpholinyl,
R³ is methyl,
R⁴ is C₁₋₇ fluoroalkyoxy, wherein R⁴ is labeled with ¹⁸F.

4. The radiolabeled compound of claim 1, wherein
R¹ and R² together with the nitrogen atom to which they are attached, form a heterocycloalkyl, preferably morpholinyl,
R³ is methyl,
R⁴ is C₁₋₇ alkyoxy, wherein R⁴ is either labeled with ³H or ¹¹C.

5. The radiolabeled compound of claim 1 selected from the group consisting of:

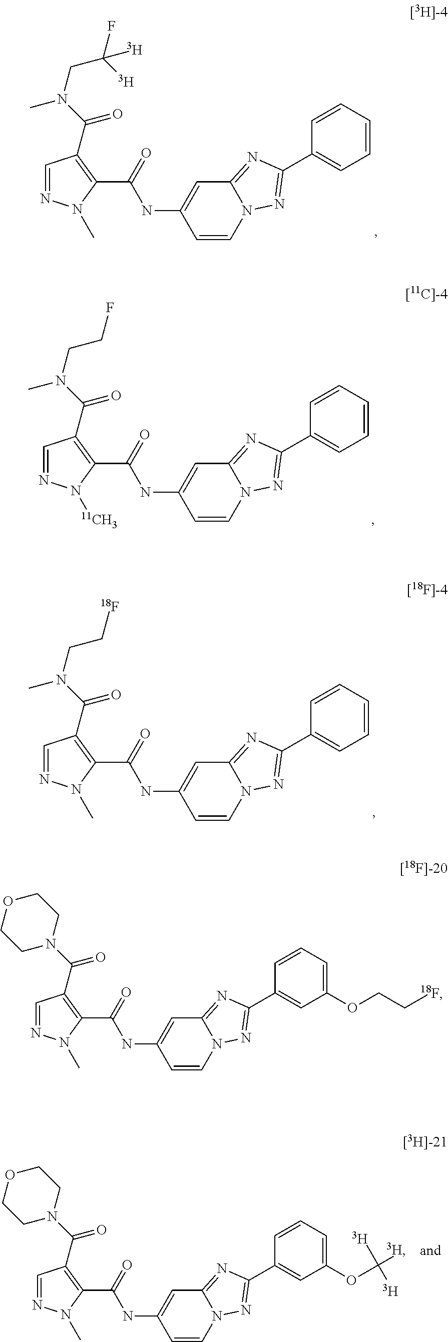

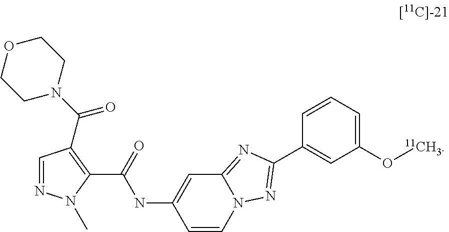

6.-9. (canceled)

10. A method for positron emission tomography (PET) imaging of PDE10A in tissue of a subject, the method comprising:
a) administering an effective amount of a compound of claim 1 to the subject,
b) allowing the compound to penetrate into the tissue of the subject; and
c) collecting a PET image of the CNS or brain tissue of the subject.

11. A method for the detection of PDE10A functionality in a tissue of a subject, the method comprising
a) administering an effective amount of a compound of claim 1 to the subject,
b) allowing the compound to penetrate into the tissue of the subject; and
c) collecting a PET image of the CNS or brain tissue of the subject.

12. (canceled)

13. A pharmaceutical composition comprising a compound as claimed in claim 1 and a pharmaceutically acceptable excipient.

14. (canceled)
